(19) United States
(12) Patent Application Publication
(10) Pub. No.: US 2006/0241541 A1
(43) Pub. Date: Oct. 26, 2006

(54) Therapeutic bandage for the heel of the foot

(76) Inventor: Sundaram Ravikumar, Briarcliff Manor, NY (US)

Correspondence Address: GORDON & JACOBSON, P.C., 60 LONG RIDGE ROAD, SUITE 407, STAMFORD, CT, 06902, US

(21) Appl. No.: 11/120,044

(22) Filed: May 2, 2005

Related U.S. Application Data
(63) Continuation-in-part of application No. 11/110,323, filed on Apr. 20, 2005.

(51) Int. Cl. A61F 13/00; A61F 15/00

(52) U.S. Cl. 602/46

(57) ABSTRACT

An improved therapeutic bandage that covers a wound (e.g., ulcer) in the heel region of the human foot includes at least one skin contacting region disposed about the periphery of the bandage. The skin-contact region has adhesive material for removably affixing the bandage to the skin. A central portion supports a hydrophilic foam sponge that is operably disposed adjacent the wound and absorbs bodily fluids that emanate from the wound. An integral air bladder is reduces pressure applied to the wound. Preferably, the foam sponge can be inserted and removed from at least one pocket defined by the central portion. The foam sponge may be loaded with a therapeutic drug.

This is a continuation-in-part of U.S. Ser. No. 11/110,323 filed Apr. 20, 2005, entitled “Compression Apparatus for Applying Localized Pressure to a Limb”, the complete disclosure of which is hereby incorporated by reference herein.

### BACKGROUND OF THE INVENTION

1. Field of the Invention

This invention relates broadly to medical devices. More particularly, this invention relates to a therapeutic bandage for protecting the heel region of the foot and/or aiding in the healing of an ulcer or like wounds in the heel region of the foot.

2. State of the Art

An ulcer is commonly defined as a lesion on the surface of the skin, or on a mucous surface, manifested through a superficial loss of tissue. Ulcers are usually accompanied by inflammation and often become chronic. Chronic ulcers are difficult to heal; they almost always require medical intervention.

Heel ulcers are common occurrences when patients are confined to a bed for a long period of time. In such cases, the heel ulcer is typically attributed to localized pressure applied to the heel, which reduces superficial blood flow in the affected region. Once necrosis of the skin occurs, the skin is opened up with various stages of tissue loss.

At the present time, there are several methods of treating heel ulcers including the use of foam sponge dressings and various splints that elevate the heel from the bed to facilitate healing. However, once necrosis of the tissue takes place, it is very difficult to achieve and maintain maximum contact of such dressings with the open wound, thereby limiting the effectiveness of such dressings in healing the ulcer.

Thus, there remains a need in the art to provide improved apparatus and methods for the treatment of heel ulcers. Additionally, many other problems, obstacles and challenges present in existing modalities for the treatment of heel ulcers will be evident to caregivers and others of experience and ordinary skill in the art. With the severe shortcomings of the prior art in mind, it is an overriding object of the present invention to improve generally over the prior art in providing a wound treatment apparatus and methodology for the heel of the foot that is simple to use and is sure to produce the desired treatment.

### SUMMARY OF THE INVENTION

It is therefore an object of the invention to provide a wound healing apparatus (and corresponding methodology) for the heel region of the foot that effectively maintains maximum contact with an open wound in the heel region of the foot.

It is another object of the invention to provide such a wound healing apparatus (and corresponding methodology) that reduces local pressures that would otherwise be applied to the open wound.

It is a further object of the invention to provide such a wound healing apparatus (and corresponding methodology) that is simple to use and is sure to produce the desired treatment.

In accord with these objects, which will be discussed in detail below, an improved therapeutic bandage that covers a wound (e.g., an ulcer) in the heel region of the human foot includes at least one skin contacting region disposed about the periphery of the bandage. The skin-contact region has adhesive material for removably affixing the bandage to the skin. A central portion of the bandage supports a hydrophilic foam sponge that is operably disposed adjacent the wound and absorbs body fluids that emanate (ooze) from the wound. An integral air bladder located behind the sponge reduces local pressures applied to the wound. Preferably, the foam sponge can be inserted and removed from at least one pocket defined by the central portion. The foam sponge may be loaded with a therapeutic drug.

According to one embodiment of the invention, the air bladder is adapted such that it lies over the wound in use.

According to another embodiment of the invention, the air bladder includes a central void that is adapted to lie over the wound in use.

Additional objects and advantages of the invention will become apparent to those skilled in the art upon reference to the detailed description taken in conjunction with the provided figures.

### BRIEF DESCRIPTION OF THE DRAWINGS

FIG. 1 is a top schematic view of a therapeutic bandage in accordance with the present invention.

FIG. 2 is a cross-sectional view of the therapeutic bandage of FIG. 1.

FIG. 3 is pictorial illustration of the therapeutic bandage of FIG. 1 affixed to a patient's foot.

FIG. 4 is a cross-sectional view of an alternate embodiment of a therapeutic bandage in accordance with the present invention.

### DETAILED DESCRIPTION OF THE PREFERRED EMBODIMENT

Referring to the figures, the present invention generally comprises a flexible therapeutic bandage 10 that is operably affixed to the skin of the patient's foot such that it covers a wound (e.g., an ulcer) in the heel region of the foot. The therapeutic bandage 10 includes at least one adhesive-based skin contacting region about the periphery of the bandage that removably affixes the bandage to the skin of the foot. A central portion of the bandage 10 supports a hydrophilic foam sponge that is operably disposed adjacent the wound where it contacts the wound (or is in close proximity thereto) and absorbs body fluids that emanate from the wound. The central portion of the bandage 10 has an air bladder integral thereto. The air bladder is adapted to reduce local pressures applied to the wound. Preferably, the adhesive-based skin contacting region(s), the foam sponge and the air bladder are sized and shaped to accommodate the curvature of the heel of the foot. Such features aid in maintaining contact of the foam sponge with the wound. They also aid in properly locating the bandage such that local pressure applied to the wound is reduced. Each of these results contributes to the healing of the wound.

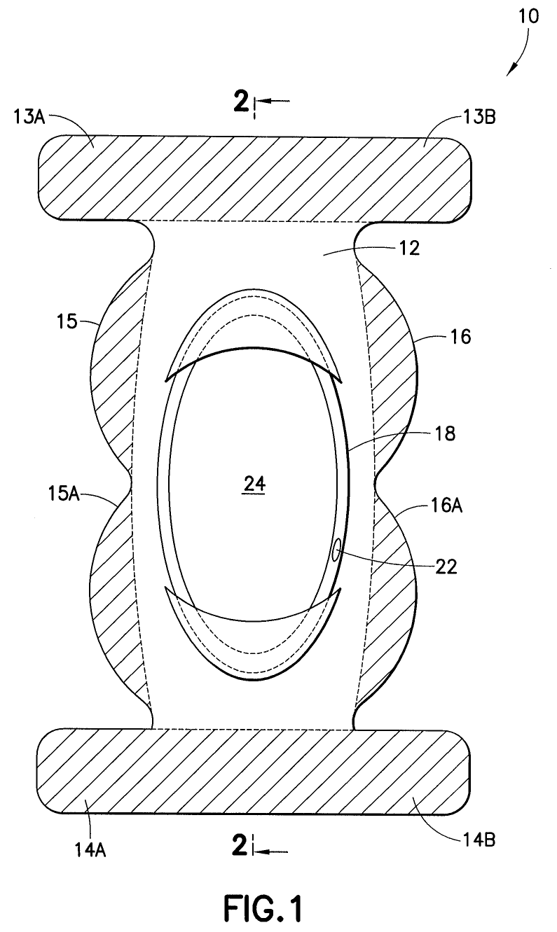

Turning now to FIG. 1, an exemplary embodiment of a therapeutic bandage 10 in accordance with the present invention includes a first sheet 12 of flexible material, which may be a semi-elastic fabric, a polymer-based film, or other suitable material, with a first set of tabs 13A, 13B that extend from one end of the sheet 12 and a second set of tabs 14A, 14B that extend from the opposite end of the sheet 12. The first set of tabs 13A, 13B are adapted to wrap around the ankle of the foot, and the second set of tabs 14A, 14B are adapted to wrap around the major portion of foot as shown in FIG. 3. The sides 15, 16 of the sheet 12 have V-cut portions 15A, 15B disposed opposite one another that accommodate the curve of the foot at the back of the heel as best shown in FIG. 3.

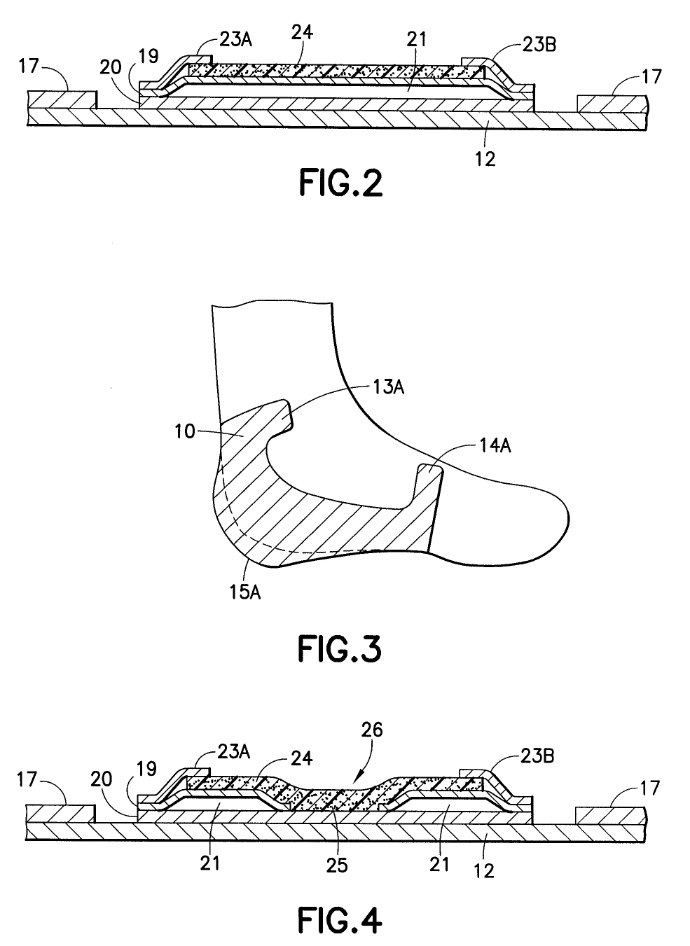

The peripheral regions of the sheet 11, which are marked with hatched lines in FIG. 1, have an adhesive layer 17 (FIG. 2) applied thereto that operably contacts the skin of the foot to thereby removably affix the bandage 10 to the skin of the foot.

The central portion of the sheet 12 supports an air bladder 18. Preferably the air bladder 18 is realized by two plastic films 19, 20 that are joined together preferably by heat or RF welding to form an air chamber 21 therebetween as shown in FIG. 2. The air bladder 18 can be manually inflated by a pump (e.g., a standard syringe barrel) that is interfaced thereto via a valve assembly 22 as shown in FIG. 1. The valve assembly 22 may be a luer having an internal valve which allows for inflation and deflation. Alternatively, both separate inflation and deflation valves may be provided. In yet another alternative, the inflation/deflation valve(s) may be omitted and the air bladder 18 pre-filled with air at the desired pressure during manufacture. The air bladder 18 acts to distribute any pressure applied to the heel over larger area of the heel (as compared to the local area of the wound) and thus reduces the local pressure applied to the wound. Where a valve is provided, the air bladder 18 may be deflated during walking if desired in order to minimize compressive forces applied to the wound during walking.

The central portion of the sheet 12 also supports a pair of flaps or pockets 23A, 23B. A hydrophilic foam sponge 24 is removably inserted into the pockets 23A, 23B such that it is supported by the sheet 12. The sponge 24 operably covers the heel and is operably disposed adjacent the wound where it contacts the wound (or is in close proximity thereto) and absorbs body fluids that emanate from the wound. The sponge 24 may be loaded with one or more therapeutic agents (such as an antibacterial agent or gel). The sponge 24 may be removed from the pockets 23A, 23B after being used and replaced with a new sponge. Alternatively, the sponge 24 can be an integral part of the bandage 10 that cannot be removed. In the illustrative embodiment shown in FIG. 3, the flaps 23A, 23B and sponge 24 are disposed together with the air bladder 18 on the skin-contacting side of the sheet 12. Alternatively, the air bladder 18 may be disposed on the opposite side of the sheet 12 (e.g., the side that faces away from the skin during use).

The size and shape of the skin contacting sheet 12 (e.g., the V-cut portions 15A, 16A), the air bladder 18 and the foam sponge 24 accommodate the curvature of the heel of the foot. For example, the air bladder 18 and/or the foam sponge 24 can be pre-formed to fit the heel of the foot. Such features aid in maintaining contact of the foam sponge with the wound. They also aid in properly locating the bandage such that local pressure applied to the wound is reduced. Each of these results contributes to the healing of the wound.

In an alternate embodiment as shown in FIG. 4, the air bladder 18 can have a central void 25 (e.g., a donut shape when viewed from above) that overlies a substantial portion of the wound. The central void 25 further reduces the local pressure applied to the wound during use. Moreover, sponge 24 may have a central depression 26 facing the wound as shown. The central depression 26 provides a reservoir for therapeutic agents/medicines that are loaded into the sponge 24.

During operation, the air bladder 18 is inflated with air (if need be). The sponge 24 may be loaded with the desired therapeutic agent(s). The bandage 10 is located such that the sponge 24 covers the wound (e.g., the ulcer) on the heel of the foot. The adhesive portions 17 are then located about the foot and pressed down such that they contact the skin of the foot, thereby affixing the bandage 10 to the heel of the foot as best shown in FIG. 3. The air bladder 18 aids in reducing local pressures applied to the wound. Preferably, the adhesive portions 17 maintain contact of the foam sponge 24 with the wound. Each of these results contributes to the healing of the wound.

There have been described and illustrated herein embodiments of a therapeutic bandage and corresponding method of operation. While particular embodiments of the invention have been described, it is not intended that the invention be limited thereto, as it is intended that the invention be as broad in scope as the art will allow and that the specification be read likewise. Thus, while particular shapes and configurations of the elements of the bandage have been disclosed as preferred, it will be appreciated that other shapes and configurations can be used as well. In addition, while the use of a pressure-reducing air chamber has been disclosed, it will be understood that other-types of fluid (such as water-based gels or liquids) can be used in a pressured chamber for pressure reduction. It will therefore be appreciated by those skilled in the art that yet other modifications could be made to the provided invention without deviating from its spirit and scope as claimed.

1. A therapeutic bandage for covering a wound in the heel region of the human foot comprising:
at least one skin contacting region disposed about the periphery of the bandage, said skin-contact region having adhesive material for removably affixing the bandage to the skin of the human foot;
a central portion that supports a hydrophilic pad that is operably disposed adjacent the wound and absorbs body fluids that emanate from the wound, wherein said central portion comprises at least one pocket that receives said pad such that said pad is removable therefrom;
an air bladder that covers the heel of the foot and reduces pressure applied to the wound, said air bladder having a chamber and a valve assembly fluidly coupled to said chamber for inflating and deflating said chamber; and
cutouts disposed about the periphery of the bandage opposite one another that accommodate the curvature of the heel of the foot.

2. (canceled)

3. A therapeutic bandage according to claim 1, wherein:
said air bladder is integral to said central portion and disposed adjacent said pad.

4. A therapeutic bandage according to claim 1, wherein:
said air bladder has a central void that is operably disposed to overlie a substantial portion of the wound.

5. A therapeutic bandage according to claim 1, wherein:
said pad has a central depression that faces the wound.

6. A therapeutic bandage according to claim 1, wherein:
said air bladder is sized to operably cover a back part and a bottom part of the heel of the foot.

7. A therapeutic bandage according to claim 1, further comprising:
a first end disposed opposite a second end;
a first set of tabs that extend from said first end, the first set of tabs being operably wrapped around the ankle of the foot and affixed thereto; and
a second set of tabs that extend from said second end, the second set of tabs being operably wrapped around the major portion of the foot and affixed thereto.

8. A therapeutic bandage according to claim 1, wherein:
said pad is loaded with a therapeutic agent.

9. A therapeutic bandage according to claim 1, wherein:
said pad comprises a foam sponge.

10. A therapeutic bandage according to claim 1, wherein:
said valve assembly comprises a valve that provides for both inflation and deflation of said chamber.

11. A therapeutic bandage according to claim 1, wherein:
said valve assembly includes at least one valve for inflation and deflation of said chamber.

12. A therapeutic bandage according to claim 1, further comprising:
an air pump that is operably coupled to said valve assembly for inflation of said chamber.
